(19) United States
(12) Patent Application Publication
(10) Pub. No.: US 2003/0004199 A1
(43) Pub. Date: Jan. 2, 2003

(54) Method for preventing or treating pulmonary inflammation by administering an endothelin antagonist

(76) Inventor: Isabelle Ounis, Mountain View, CA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/121,039

(22) Filed: Apr. 11, 2002

Related U.S. Application Data
(60) Provisional application No. 60/283,304, filed on Apr. 12, 2001.

(51) Int. Cl. A61K 31/422

(52) U.S. Cl. 514/374

(57) ABSTRACT

Prevention or treatment of disorders of chronic or acute pulmonary inflammation by administration of an endothelin antagonist.

### FIELD OF THE INVENTION

The present invention relates to the prevention or treatment of disorders of chronic or acute pulmonary inflammation by administering an endothelin antagonist to a subject in need thereof.

### BRIEF DESCRIPTION OF THE INVENTION

Endothelin is a potent vasoconstrictor. The endothelin (ET) ET-A and ET-B receptor subtypes are found, inter alia, in human lung, predominantly in airway smooth muscle, and vascular smooth muscle and, to a lesser extent nerves (see, e.g., Clin Exp Pharmacol Physiol: 26(2): 168-171, 1999). ET-1, ET-2, ET-3 and ET-B have been shown to cause bronchoconstriction (see, e.g., European Journal of Pharmacology: 191; 319-328, 1990). Patients suffering from chronic obstructive pulmonary disease (COPD, which is a disorder of chronic pulmonary inflammation), have been shown to have elevated levels of sputum ET-1 (see, e.g., European Respiratory Journal: 13(6); 1288-1292, 1999). Additionally, plasma levels of ET-1 are elevated in COPD patients during attacks (see, e.g., Chinese Journal of Tuberculosis: 16(5); 287-289, 1993). COPD patients additionally excrete higher amounts of ET-1 compared to healthy subjects (see e.g., Respiration: 61(5); 263-268, 1994). As reported by C. Hogg et al., Am. J. Respir. Cell. Biol. Vol. 23, pp.404-410 (2000) ET-A antagonists have demonstrated an anti-inflammatory profile on leukocyte driven inflammation. Accordingly, molecules that prevent or inhibit the production of ET can be expected to be useful to prevent and treat disorders of chronic or acute pulmonary inflammation such as COPD and adult respiratory distress syndrome (ARDS).

Endothelin antagonists, which are compounds capable, inter alia, of inhibiting the binding of endothelin peptides to endothelin receptors, are useful in the treatment of endothelin-related disorders. While certain such compounds have been described as having utility in the treatment of hypertension, the present invention provides a method employing these compounds specifically for the treatment of chronic or acute pulmonary inflammation.

### DETAILED DESCRIPTION OF THE INVENTION

The present invention provides a method for the prevention or treatment of chronic or acute pulmonary inflammation in a mammal, comprising administering an endothelin antagonist to said mammal in an amount effective therefor.

The endothelin antagonist employed may be any compound capable of inhibiting the action of endothelin peptides, especially, endothelin-1 (ET-1), endothelin-2 (ET-2) and/or endothelin-3 (ET-3). The endothelin antagonists described in the following documents, incorporated herein by reference in their entirety, are exemplary of those contemplated for use in the present method: U.S. Pat. No. 5,378,715; U.S. Pat. No. 5,514,696; U.S. Pat. No. 5,420,123; U.S. application Ser. No. 114,251, filed Aug. 30, 1993; U.S. application Ser. No. 08/728,238, filed Oct. 8, 1996; European Patent Application 702,012; U.S. application Ser. No. 08/754,715, filed Nov. 21, 1996; U.S. application Ser. No. 08/692,869, filed Jul. 25, 1996; U.S. Application Serial No. 60/011,974, filed Feb. 20, 1996; U.S. Application Serial No. 60/013,491, filed Mar. 12, 1996; U.S. Application Serial No. 60/015,072, filed Apr. 9, 1996; World Patent Application 94/27979; U.S. Pat. No. 5,543,521; U.S. Pat. No. 5,464,853; U.S. Pat. No. 5,514,691; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; U.S. Pat. No. 5,334,598; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016; GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ET_{A} Receptor”, Life Sciences, Vol. 50(4), pp. 247-255 (1992)); PD 156707 (Reynolds, E., et al., “Pharmacological Characterization of PD 156707, an Orally Active ET_{A} Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”, The Journal of Pharmacology and Experimental Therapeutics, Vol. 275(3), pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91, pp. 8052-8056 (1994)); SB 217242 (Ohlstein, E. H., et al., “Nonpeptide Endothelin Receptor Antagonists. VI:Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-127722 (Opgenorth, T. J., et al., “Pharmacological Characterization of A-127722: An Orally Active and Highly Potent ET_{A}-Selective Receptor Antagonist”, The Journal of Pharmacology and Experimental Therapeutics, Vol. 276(2), pp.473-481 (1996)); TAK-044 (Masuda, Y., et al., “Receptor Binding and Antagonist Properties of a Novel Endothelin Receptor Antagonist, TAK-044 {Cyclo[D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human Endothelin_{A} and Endothelin_{B} Receptors”, The Journal of Pharmacology and Experimental Therapeutics, Vol. 279(2), pp. 675-685 (1996)); bosentan (Ro 47-0203, Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)); and TBC-11251, i.e.:

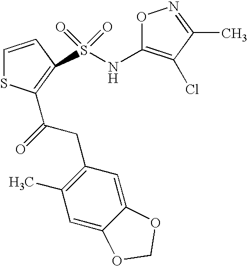

(IBC International Conference on Endothelin Inhibitors, Coronado, Calif. (February 1996) and 211th American Chemical Society National Meeting, New Orleans, La. (March 1996)). These exemplary compounds may, for example, be prepared by methods, and employed at dosages, such as those described in the aforementioned documents.

Endothelin antagonists containing a sulfonamide moiety (—SO₂—NH—) are preferred, particularly those described in U.S. Pat. No. 5,612,359. and U.S. Pat. No. 6,043,265. Especially is the following compound:

N-[[2′-[[(4,5-dimethyl-3-isoxazolyl)amino]sulfonyl]-4-(2-oxazolyl)[,1′-biphenyl]-2-yl]methyl]-N,3,3-trimethylbutanamide, having the structure:

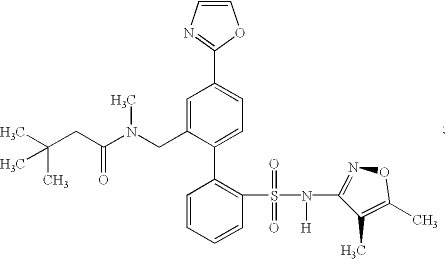

and pharmaceutically acceptable salts thereof. These preferred endothelin antagonists, and particularly the especially preferred compound shown above, are described as having a number of utilities such as the treatment of congestive heart failure and hypertension (see e.g., the disclosures in U.S. Pat. No. 5,612,359 and U.S. Application Serial No. 60/035,832,) wherein the complete recitation of all these utilities is incorporated herein by reference; these preferred endothelin antagonists may be employed for each of these utilities alone or in combination with an agent such as an angiotensin II (AII) receptor antagonist (including irbesartan, 2-n-butyl-4-spirocyclopentane-1-[(2′-(tetrazol-5-yl)biphenyl-4-yl)methyl]-2-imidazolin-5-one).

The mammal may be any mammal subject to this malady, especially, a human. The endothelin antagonist may be administered in any suitable manner such as orally or parenterally, in an effective amount, such as within a dosage range of about 0.1 to about 100 mg/kg, preferably about 0.2 to about 50 mg/kg and more preferably about 0.5 to about 25 mg/kg (or from about 1 to about 2500 mg, preferably from about 5 to about 2000 mg) in single or 2 to 4 divided daily doses.

The present invention also provides pharmaceutical compositions for the prevention or treatment of pain, comprising an endothelin antagonist in an amount effective therefor and a pharmaceutically acceptable vehicle or diluent. The endothelin antagonist can be utilized in a composition such as an inhaled aerosol, tablet, capsule, sterile solution or suspension, compounded in a conventional manner with a physiologically acceptable vehicle or carrier, excipient, binder, preservative, stabilizer, flavor, etc. as called for by accepted pharmaceutical practice.

In the methods and compositions of the present invention, the endothelin antagonist may, for example, be employed alone, in combination with one or more other endothelin antagonists, or with another compound useful for the treatment of chronic or acute pulmonary inflammation, such as bronchodilators, antibiotics, and anti-inflammatory compounds (including PDE4 inhibitors and TNF alpha inhibitors). Additionally, the endothelin antagonists of the present invention may be employed in combination with one or more other compounds useful in the treatment of endothelin-associated disorders. For example, the compounds of this invention can be formulated in combination with endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abciximab, eptifibatide, and tirofiban), P2Y(AC) antagonists (e.g., clopidogrel, ticlopidine and CS-747), and aspirin; anticoagulants such as warfarin, low molecular weight heparins such as enoxaparin, Factor VIIa inhibitors, and Factor Xa inhibitors such as those described in U.S. Ser. No. 09/496,571 filed Feb. 2, 2000 (attorney docket HA 723); renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase inhibitors such as pravastatin, lovastatin, atorvastatin, simvastatin, NK-104 (a.k.a. itavastatin, or nisvastatin or nisbastatin) and ZD-4522 (a.k.a. rosuvastatin, or atavastatin or visastatin); squalene synthetase inhibitors; fibrates; bile acid sequestrants such as questran; niacin; anti-atherosclerotic agents such as ACAT inhibitors; MTP inhibitors such as those described in U.S. Ser. No. 09/007,938 filed Jan. 16, 1998 (attorney docket HX 91); calcium channel blockers such as amlodipine besylate; potassium channel activators; alpha-adrenergic agents, beta-adrenergic agents such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide or benzothiazide as well as ethacrynic acid, tricrynafen, chlorthalidone, furosemide, musolimine, bumetanide, triamterene, arniloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), biguanide/glyburide combinations such as those described in U.S. Ser. No. 09/432,465 filed Nov. 3, 1999 (attorney docket LA 46) and U.S. Ser. No. 09/460,920 filed Dec. 14, 1999 (attorney docket LA 46a); thiozolidinediones (e.g. troglitazone, rosiglitazone and pioglitazone), and PPAR-gamma agonists; mineralocorticoid receptor antagonists such as spironolactone and eplerenone; growth hormone secretagogues such as those described in U.S. Ser. No. 09/417,180 filed Oct. 12, 1999 (attorney docket LA 25) and U.S. Ser. No. 09/506,749 filed Feb. 18, 2000 (attorney docket LA 26); aP2 inhibitors such as those described in U.S. Ser. No. 09/391,053 filed Sep. 7, 1999 (attorney docket LA 24a) and U.S. Ser. No. 09/390,275 filed Sep. 7, 1999 (attorney docket LA 24b); digitalis; ouabian; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin); cyclosporins; steroids such as prednisone or dexamethasone; gold compounds; cytotoxic drugs such as azathiprine and cyclophosphamide; TNF-alpha inhibitors such as tenidap; anti-TNF antibodies or soluble TNF receptor such as etanercept (Enbrel) rapamycin (sirolimus or Rapamune), leflunimide (Arava); and cyclooxygenase-2 (COX-2) inhibitors such as celecoxib (Celebrex) and rofecoxib (Vioxx).

What is claimed is:

1. A method for preventing or treating disorders of chronic or acute pulmonary inflammation in a mammal, comprising administering to said mammal the compound N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide or a salt thereof in an amount effective therefor.

2. The method of claim 1, wherein said mammal is a human.

3. The method of claim 1 wherein said disorder is COPD.

4. The method of claim 1 wherein said disorder is ARDS.

5. A pharmaceutical composition for the prevention or treatment of disorders of chronic or acute pulmonary inflammation in a mammal, comprising the compound N-[[2′-[[(4,5-dimethyl-3-isoxazolyl)amino]sulfonyl]-4-(2-oxazolyl)[1,1′-biphenyl]-2-yl]methyl]-N,3,3-trimethylbutanamide or a salt thereof in an amount effective therefor and a pharmaceutically acceptable vehicle or diluent.
